(19) United States
(12) Patent Application Publication
(10) Pub. No.: US 2022/0117801 A1
(43) Pub. Date: Apr. 21, 2022

(54) SUBLINGUAL DENTAL ABSORBENT PAD

(71) Applicant: MDT Micro Diamond Technologies, LTD, Afula (IL)

(72) Inventors: Patrick William Strane, Woodstock, GA (US); Christie Wilkins, Acworth, GA (US); Cody Sunderland, Woodstock, GA (US)

(73) Assignee: MDT Micro Diamond Technologies, LTD, Afula (IL)

(21) Appl. No.: 17/071,657

(22) Filed: Oct. 15, 2020

(51) Int. Cl. A61F 13/20

(52) U.S. Cl. CPC A61F 13/2008; A61F 13/2022; A61F 13/2068

(57) ABSTRACT

A sublingual dental absorbent pad has a U-shaped body made of a moisture permeable bottom layer, a moisture impermeable top layer, an a moisture absorbent middle layer. The dental absorbent pad has a generally U-shape so that it fits in the mouth under the tongue. The dental absorbent pad has a central portion from which extend two legs which are separated from each other in a divergent manner to form a V-shaped space therebetween. The central portion has a front edge with a generally flat or shallow arcing configuration.

### TECHNICAL FIELD

This invention relates generally to dental absorbent pads, and particularly to sublingual dental absorbent pads which are used in the dental industry.

### BACKGROUND OF THE INVENTION

Small absorbent pads are often used in dentistry to absorb saliva from the mouth of a patient during a dental procedure. These pads are typically positioned between the teeth and cheek or between the teeth and tongue and are replaced when they become saturated.

The most common type of dental absorbent pad is simply a mass or roll of cotton. While these cotton rolls may provide absorbency, they typically expand with moisture and may become difficult to properly position.

The most effective dental absorbent pads in terms of absorption volume typically have a moisture permeable outer layer made of a nylon fabric, a super absorbent polymer core, and a moisture impermeable, second outer layer of polyethylene film. These materials are held together with a layer of adhesive. This adhesive however causes the peripheral edges of the pad to be stiff and abrasive, which may cause irritation to the tissue of the mouth.

Another type of dental absorbent pad is shown in patent publication WO2014003642 A1. Here the absorbent pad has a liquid transporting wrapping which encloses a superabsorbent material. The superabsorbent material is typically a sheet of cotton material which includes granules of a superabsorbent polymer embedded within the cotton material. A problem with this type of device is that the granules of superabsorbent polymer is released by the cotton material and migrates through the liquid transporting wrapping. Thus, the superabsorbent polymer grains are released into the mouth of the patient. This product also is restricted in shape to generally that of the sheet of cotton material which provides the structural integrity of the dental absorbent pad and may not conform with an individual's mouth.

Dental pads have also been designed having a generally semi-circular shape. However, these pads typically overlay the bottom teeth, causing them to occlude visual and physical access to the bottom teeth.

Yet another problem with prior art dental pads is their inability to remain anchored in position. The prior art dental pads may slip from their intended position, thereby becoming less effective in absorbing saliva.

Accordingly, it is seen that a need remains for a dental absorbent pad which is more comfortable or conformable for a patient while also allowing visual and physical access to the lower teeth. It is to the provision of such that the present invention is primarily directed.

### SUMMARY OF THE INVENTION

In a preferred form of the invention, a sublingual dental absorbent pad comprises a U-shaped body and a layer of adhesive coupled to the U-shaped body. The U-shaped body has a central portion and two legs extending from the central portion. The U-shaped body has a moisture permeable bottom layer, a moisture impermeably top layer, and a moisture absorbent middle layer positioned between the bottom layer and the top layer.

In another preferred from of the invention, a sublingual dental absorbent pad comprises a first body having a moisture permeable bottom layer, a top layer, and a moisture absorbent middle layer positioned between the bottom layer and said top layer, and a second body coupled to the first body along a bend line.

### BRIEF DESCRIPTION OF THE DRAWING

FIG. 1 is a perspective view of a sublingual dental absorbent pad in a preferred form of the invention.

FIG. 2 is a perspective, exploded view of the sublingual dental absorbent pad of FIG. 1.

FIG. 3 is a top view of the sublingual dental absorbent pad of FIG. 1.

FIG. 4 is a top view of the sublingual dental absorbent pad of FIG. 1, shown positioned within the lower mouth of a wearer.

FIG. 5 is a perspective view of a sublingual dental absorbent pad in another preferred form of the invention.

FIG. 6 is a perspective view of the sublingual dental absorbent pad of FIG. 5, shown in a bent configuration.

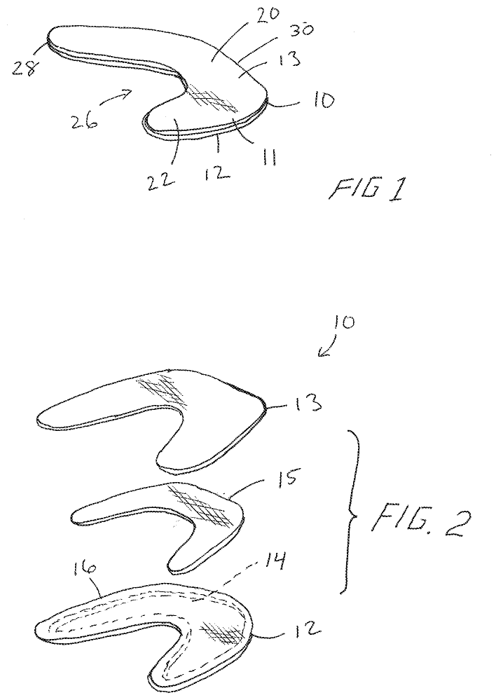

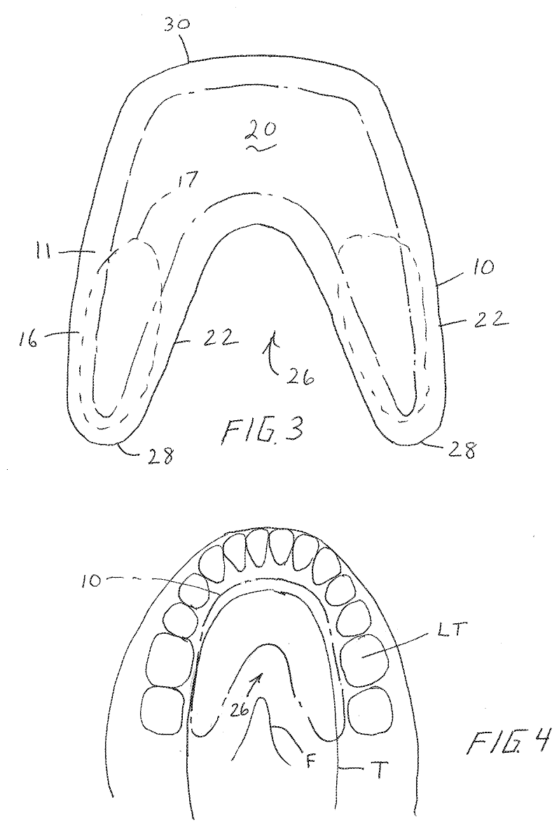

### DETAILED DESCRIPTION

With reference next to the drawings, there is shown a sublingual dental absorbent pad 10 embodying principles of the invention in a preferred form. The pad 10 has a C-shaped or U-shaped body 11 made of a moisture permeable bottom layer 12, a moisture impermeable top layer 13, and a moisture absorbent middle layer 15 positioned between the top layer 13 and bottom layer 12. The top layer 13 is bonded to the bottom layer 12 along a peripheral margin 16, the bonding may be accomplished through any conventionally know means such as heat bonding, adhesive bonding, or the like. The bottom layer 12 forms a recess or depression 14 in which the middle layer 15 resides. Portions of the bottom surface of the bottom layer 12 may include a pressure activated adhesive layer 17.

The bottom layer 12 is preferably a fabric mesh material, such as the micro-thin high density polyethylene woven or non-woven netting, such as the 0.11 mm non-woven film model number DELNET P530 HDPE sold by Delstar Technologies, Inc. of Middletown, Del. The top layer 13 is preferably a moisture non-permeable plastic, such as a polypropylene sheet material. The moisture absorbent middle layer 15 is preferably a sheet or layer of super absorbent polymer such as a sodium sulfite. The pressure activated adhesive layer 17 may be medical grade, rubber based adhesive.

The dental absorbent pad 10 has a generally U-shape to reside in the mouth under the tongue T and between the two opposing sets of lower teeth (sublingual or sublingual mucosa region) to provide better comfort than the semi-circular shape of the prior art pads while also providing access to the bottom teeth as the dental absorbent pad 10 is recessed below the top level of the teeth rather than being positioned over the teeth as with the dental pads of the prior art.

The dental absorbent pad has a central portion 20 from which two tails, legs, arms, extensions, or projections 22. The legs 22 are separated from each other in a divergent manner from the central portion 20 so as to form a V-shaped space 26 therebetween. The V-shaped space 26 allows the wearer's frenulum F of the tongue (lingual frenulum) to fit therein for better fitting of the dental absorbent pad 10. Each leg 22 is tapered or narrows as it extends outwardly to its rounded end 28 so as to provide comfort within the mouth as this area of the mouth between the tongue and teeth narrows as it approaches the back of the mouth. The bottom layer 12 of the legs 22 is provided with the adhesive layer 17.

The central portion 20 has a front edge 30 with a generally flat or slightly arcing (a shallow or small degree of curvature), which may also be referenced herein as a blunt front edge. It has been discovered that a generally rounded front edge, which would appear to conform with the front, bottom teeth, is actually and surprisingly hard to fit within the mouth in a position under the tongue and against the front, bottom teeth. The rounded front edge has also been discovered to cause a dental absorbent pad positioned in the sublingual mucosa area to easily pop out of position. It is believed that a front edge having a slight or shallow arc (arcuate) with a radius between 100 and 130 mm is preferred and provides for maintaining a proper fit to prevent dislodging the dental absorbent pad 10 through even small tongue motions. This shallow or slight arc may also be considered to be a “flattened” area or “substantially flat” as it includes only a slight or shallow arc.

In use, the dental absorbent pad 10 is placed below the tongue with the front edge 30 of the central portion 20 positioned generally against the front teeth and the legs 22 positioned between and against the side teeth of the lower jaw, which is known as the sublingual mucosa. This placement allows the dental absorbent pad 10 to overlay the sublingual and submandibular glands which release saliva from this region. The moisture permeably bottom layer 12 allows the passage of saliva therethrough so that it may be absorbed and contained by the moisture absorbent middle layer 15.

It should be understood that as the margin 15 area of the pad is formed from very thin and flexible layers of material which is heat welded, or otherwise welded or bonded, and is not considered to be stiff, as opposed to prior art pads. Furthermore, the reduction in stiffness and thickness of this peripheral area reduces the abrasiveness of the peripheral edge against the mouth tissues, thereby further increasing the comfort of the present invention over prior art dental absorbent pads.

It should also be understood that the dental absorbent pad 10 may be bent longitudinally and centrally so that the dental absorbent pad is arcuate. This bending of the dental absorbent pad 10 may aid in anchoring the dental absorbent pad beneath the tongue.

It should be understood that the composition of the material is not intended to be limited by those recited herein and may be different from that specifically used in the preferred embodiment. For example, the bottom layer 12 may be made of a woven, non-woven or porous polymer material, such as a woven nylon or non-woven fiber.

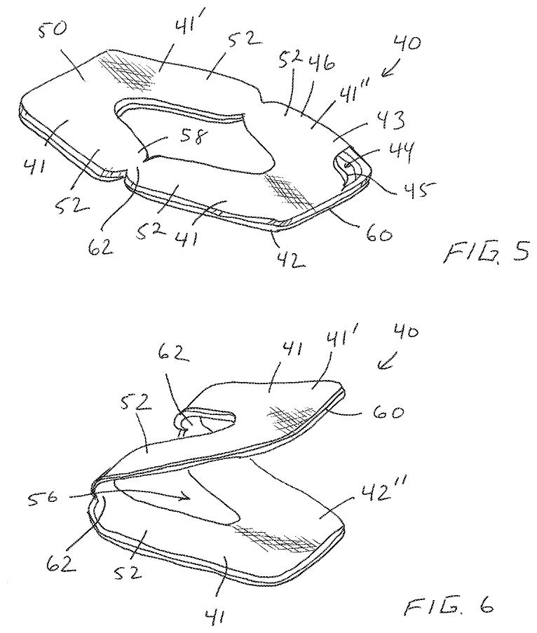

With reference next to FIGS. 5 and 6, there is a shown a dental absorbent pad 40 embodying principles of the invention in another preferred form. The pad 40 has two joined C-shaped or U-shaped bodies 41 made of a moisture permeable bottom layer 42, a moisture impermeable top layer 43, and a moisture absorbent middle layer 45 positioned between the top layer 43 and bottom layer 42. The top layer 43 is bonded to the bottom layer 42 along a peripheral margin 46, the bonding may be accomplished through any conventionally know means such as heat bonding, adhesive bonding, or the like. The bottom layer 42 forms a recess or depression 44 in which the middle layer 45 resides. Portions of the bottom surface of the bottom layer 42 may include a pressure activated adhesive layer.

The bottom layer 42 is preferably a fabric mesh material, such as the micro-thin high density polyethylene woven or non-woven netting, such as the 0.11 mm non-woven film model number DELNET P530 HDPE sold by Delstar Technologies, Inc. of Middletown, Del. The top layer 43 is preferably a moisture non-permeable plastic, such as a polypropylene sheet material. The moisture absorbent middle layer 45 is preferably a sheet or layer of super absorbent polymer such as a sodium sulfite. The pressure activated adhesive layer may be medical grade, rubber based adhesive.

The two U-shaped bodies 41 are joined to each other at a laterally extending bite, pivot area, bend line, or bend 62 located at the rounded ends 58 of the legs 52 of each U-shaped body 41.

When the dental absorbent pad 40 is bent along bend 62, as shown in FIG. 6, the dental absorbent pad 40 has a generally doubled U-shape to reside in the mouth under the tongue T and between the two opposing sets of lower teeth (sublingual or sublingual mucosa region) to provide better comfort than the semi-circular shape of the prior art pads while also providing access to the bottom teeth as the dental absorbent pad 40 is recessed below the top level of the teeth rather than being positioned over the teeth as with the dental pads of the prior art.

Each U-shaped body 41 has a central portion 50 from which two tails, legs, arms, extensions, or projections 52. The legs 52 are separated from each other in a divergent manner from the central portion 50 so as to form a V-shaped space 56 therebetween. The V-shaped space 56 allows the wearer's frenulum F of the tongue (lingual frenulum) to fit therein for better fitting of the dental absorbent pad 40. Each leg 52 is tapered or narrows as it extends outwardly to its rounded end 58 so as to provide comfort within the mouth as this area of the mouth between the tongue and teeth narrows as it approaches the back of the mouth. The bottom layer 42 of the legs 52 of one U-shaped body 41 is provided with the adhesive layer.

Each U-shaped body central portion 50 has a front edge 60 with a generally flat or slightly arcing (a shallow or small degree of curvature), which may also be referenced herein as a blunt front edge. It has been discovered that a generally rounded front edge, which would appear to conform with the front, bottom teeth, is actually and surprisingly hard to fit within the mouth in a position under the tongue and against the front, bottom teeth. The rounded front edge has also been discovered to cause a dental absorbent pad positioned in the sublingual mucosa area to easily pop out of position. It is believed that a front edge having a slight or shallow arc with a radius between 100 and 130 mm is preferred and provides for maintaining a proper fit to prevent dislodging the dental absorbent pad 40 through even small tongue motions. This shallow or slight arc may also be considered to be a “flattened” area or “substantially flat” as it includes only a slight or shallow arc.

In use, the dental absorbent pad 40 is bent along the lateral bend 62 so that the two U-shaped bodies 41 generally overlay each other. The material that forms the dental absorbent pad 40 provides an outward biasing or spring force against the bending action when bent, i.e., the two U-shaped bodies are biased away from each other along the bend 62. The dental absorbent pad 40 is then placed below the tongue with the front edge 60 of the central portion 50 positioned generally against the front teeth and the legs 52 positioned between and against the side teeth of the lower jaw, which is known as the sublingual mucosa. This placement allows the dental absorbent pad 40 to overlay the sublingual and submandibular glands which release saliva from this region. The moisture permeably bottom layer 42 allows the passage of saliva therethrough so that it may be absorbed and contained by the moisture absorbent middle layer 45. The doubling or bending of the U-shaped bodies 41 increases the saliva absorbent capacity of the dental absorbent pad 40.

The bending of the dental absorbent pad 40 along the lateral bend 62 also creates a spring effect between the two U-shaped bodies 41, between the upper U-shaped body 41′ and the lower U-shaped body 41″. This spring effect when combined with the positioning under and against the tongue causes or biases the lower U-shaped body 41″ against the bottom of the mouth, thus anchoring the dental absorbent pad 40 in place to prevent movement. To increase the spring effect, the dental absorbent pad 40 may include an additional resilient layer or may be made of thicker layers to increase the resistance to bending.

It should be understood that as the margin 45 area of the pad is formed from very thin and flexible layers of material which is heat welded, or otherwise welded or bonded, and is not considered to be stiff, as opposed to prior art pads. Furthermore, the reduction in stiffness and thickness of this peripheral area reduces the abrasiveness of the peripheral edge against the mouth tissues, thereby further increasing the comfort of the present invention over prior art dental absorbent pads.

It should be understood that the composition of the material is not intended to be limited by those recited herein and may be different from that specifically used in the preferred embodiment. For example, the bottom layer 42 may be made of a woven, non-woven or porous polymer material, such as a woven nylon or non-woven fiber.

It thus is seen that a dental absorbent pad is now provided which is more comfortable for a patient while allowing for visual and physical access to the lower teeth. And though the invention has been shown and described in its preferred form, it should be understood that additions, deletions and modifications may be made without departure from the spirit and scope of the invention as set forth in the following claims.

1. A sublingual dental absorbent pad comprising,
a U-shaped body and a layer of adhesive coupled to said U-shaped body,
said U-shaped body having a central portion and two legs extending from said central portion,
said U-shaped body having a moisture permeable bottom layer, a moisture impermeably top layer, and a moisture absorbent middle layer positioned between said bottom layer and said top layer.

2. The sublingual dental absorbent pad of claim 1 wherein said legs diverge from each other as said legs extend from said central portion.

3. The sublingual dental absorbent pad of claim 1 wherein each leg of said two legs terminates at a rounded end.

4. The sublingual dental absorbent pad of claim 1 wherein said two legs form a V-shaped space between said two legs.

5. The sublingual dental absorbent pad of claim 1 wherein each said leg of said two legs tapers as said leg extends from said central portion.

6. The sublingual dental absorbent pad of claim 1 wherein said body has a front edge oppositely disposed from said two legs, and wherein said front edge has a flat configuration.

7. The sublingual dental absorbent pad of claim 1 wherein said body has a front edge oppositely disposed from said two legs, and wherein said front edge has a shallow arcuate configuration.

8. The sublingual dental absorbent pad of claim 1 further comprising a second U-shaped body pivotally coupled to said U-shaped body.

9. The sublingual dental absorbent pad of claim 8 wherein said second U-shaped body is pivotally coupled to said U-shaped body at a junction located at said end of said two legs of said U-shaped body distally from said central portion of said U-shaped body.

10. A sublingual dental absorbent pad comprising,
a first central portion and first two legs extending from said first central portion, said first central portion and said first two legs having a moisture permeable bottom layer, a top layer, and a moisture absorbent middle layer positioned between said bottom layer and said top layer.

11. The sublingual dental absorbent pad of claim 10 wherein said first two legs diverge from each other as said first two legs extend from said central portion.

12. The sublingual dental absorbent pad of claim 10 wherein each leg of said first two legs terminates at a rounded end.

13. The sublingual dental absorbent pad of claim 10 wherein said first two legs form a V-shaped space between said first two legs.

14. The sublingual dental absorbent pad of claim 10 wherein each said leg of said first two legs tapers as said legs extend from said central portion.

15. The sublingual dental absorbent pad of claim 10 wherein said first central portion has a front edge oppositely disposed from said first two legs, and wherein said front edge has a flat configuration.

16. The sublingual dental absorbent pad of claim 10 further comprising a second central portion and second two legs extending from said second central portion pivotally coupled to said first two legs.

17. A sublingual dental absorbent pad comprising,
a first body having a moisture permeable bottom layer, a top layer, and a moisture absorbent middle layer positioned between said bottom layer and said top layer;
a second body coupled to said first body along a bend line.

18. The sublingual dental absorbent pad of claim 17 wherein said second body has a moisture permeable bottom layer, a top layer, and a moisture absorbent middle layer positioned between said bottom layer and said top layer.

19. The sublingual dental absorbent pad of claim 17 wherein said first body is a first U-shaped body having a first central portion with a first front edge and two first legs extending from said first central portion oppositely from said first front edge.

20. The sublingual dental absorbent pad of claim 19 wherein said second body is a second U-shaped body having a second central portion with a second front edge and two second legs extending from said second central portion oppositely from said second front edge.

21. The sublingual dental absorbent pad of claim 20 wherein said two second legs are pivotally coupled to said two first legs.
